(19) United States
(12) Patent Application Publication
(10) Pub. No.: US 2018/0070887 A1
(43) Pub. Date: Mar. 15, 2018

(54) BLOOD PRESSURE MEASURING DEVICE AND BLOOD PRESSURE MEASURING METHOD USING THE SAME

(71) Applicant: GWANGJU INSTITUTE OF SCIENCE AND TECHNOLOGY, Gwangju (KR)

(72) Inventors: Boreom LEE, Gwangju (KR); Jongin KIM, Gwangju (KR); Kwangjin LEE, Gwangju (KR); Dongrae CHO, Gwangju (KR)

(21) Appl. No.: 15/690,103

(22) Filed: Aug. 29, 2017

(30) Foreign Application Priority Data
Aug. 29, 2016 (KR) 10-2016-0110156
Dec. 12, 2016 (KR) 10-2016-0168285

(51) Int. Cl. A61B 5/00; A61B 5/021; A61B 5/026

(52) U.S. Cl. CPC A61B 5/7278; A61B 5/02125; A61B 5/0004; A61B 5/7225; A61B 5/02416; A61B 5/6898; A61B 5/742; A61B 5/0261; A61B 5/7203

(57) ABSTRACT

There is provided a blood pressure measuring device comprising: a first heart rate measuring module configured to measure a first heart rate at a first location of an examinee's body; a second heart rate measuring module configured to measure a second heart rate at a second location of the examinee's body, wherein the first location is different from the second location, wherein a first spacing between the first location and a heart of the examinee is different from a second spacing between the second location and the heart of the examinee; and a blood pressure estimation module configured to estimate a blood pressure of the examinee based on the first and second heart rates measured by the first and second heart rate measuring modules.

### CROSS-REFERENCE TO RELATED APPLICATIONS

This application claims the benefit of Korean Patent Application No. 10-2016-0110156, filed on Aug. 29, 2016 and Korean Patent Application No. 10-2016-0168285 filed on Dec. 12, 2016, the entire contents of which are incorporated herein by reference for all purposes as if fully set forth herein.

### BACKGROUND

### Field of the Present Disclosure

The present disclosure relates to a blood pressure measuring device and a blood pressure measuring method using the blood pressure measuring device. More particularly, the present disclosure relates to a blood pressure measuring device and a blood pressure measuring method using the blood pressure measuring device, wherein the blood pressure of an examinee is estimated based on an image of a facial portion of the examinee and an image of a finger thereof.

### Discussion of Related Art

The human physiological signal includes information indicating the health state of the person. Therefore, by measuring a physiological signal of a human, the current state of health of the person can be known. One of the widely measured physiological signals for this purpose is a blood pressure.

In the clinical aspect, the blood pressure is an indicator of the abnormalities of the circulatory system, including the heart and blood vessels. Therefore, if the measured blood pressure value is not normal, the cause of the abnormality may be grasped and appropriate treatment may be performed accordingly.

The blood pressure changes with heart rate. When the ventricles contract, blood is supplied into the arteries. The blood pressure measured at this time is called systolic blood pressure. When the ventricles expand, blood is not supplied into the arteries. The blood pressure measured at this time is called the diastolic blood pressure. Despite the fact that the blood pressure is not supplied into the arteries when the ventricles expand, the diastolic blood pressure does not become zero since the walls of the blood vessels are resilient and thus pressurizing the blood.

At hospitals and medical institutions, it may be less likely to measure the blood pressure under conditions and circumstances that may measure baseline blood pressure. However, in the case of a home, the efforts of the subject or family member can create conditions and environments that may measure the baseline blood pressure. Therefore, there has been a need for home electronic blood pressure measuring devices that family members can easily handle.

Accordingly, various researches have been conducted on the blood pressure measuring apparatus which can be easily manipulated by the public. In particular, automated blood pressure measurement devices that can measure the blood pressure indirectly are commonly developed by the development of the electronics industry.

Current automated blood pressure measuring instruments (hereinafter referred to as conventional blood pressure measuring instruments) employ the blood pressure measurement method using a volume oscillometric method which does not require a special conversion device or a microphone. In the blood pressure measuring device using the volume oscillometric method, a cuff is worn by the user. Therefore, the conventional blood pressure measuring device has a relatively large volume, is difficult to carry. Further, the cuff has to be worn every time the blood pressure is measured, which is a cumbersome.

### PRIOR ART DOCUMENT

### Patent Literature

Patent Document 1: Korean Patent No. 10-1366809 tilted as “Blood pressure measuring device and blood pressure measuring method”

### SUMMARY

This Summary is provided to introduce a selection of concepts in a simplified form that are further described below in the Detailed Description. This Summary is not intended to identify all key features or essential features of the claimed subject matter, nor is it intended to be used alone as an aid in determining the scope of the claimed subject matter.

The present disclosure is to provide a blood pressure measuring device and a blood pressure measuring method using the blood pressure measuring device, wherein the blood pressure of an examinee is estimated based on an image of a facial portion of the examinee and an image of a finger thereof, wherein a spacing between the finger and a heart of the examinee is different from a spacing between the facial portion and a heart of the examinee.

In a first aspect of the present disclosure, there is provided a blood pressure measuring device comprising: a first heart rate measuring module configured to measure a first heart rate at a first location of an examinee's body; a second heart rate measuring module configured to measure a second heart rate at a second location of the examinee's body, wherein the first location is different from the second location, wherein a first spacing between the first location and a heart of the examinee is different from a second spacing between the second location and the heart of the examinee; and a blood pressure estimation module configured to estimate a blood pressure of the examinee based on the first and second heart rates measured by the first and second heart rate measuring modules.

In one implementation of the first aspect, the first location corresponds to a finger of the examinee.

In one implementation of the first aspect, the first heart rate measuring module includes: a first camera for imaging the first location to obtain a first image; and a first heart rate calculation unit for calculating the first heart rate of the examinee based on the first image imaged from the first camera.

In one implementation of the first aspect, the first heart rate calculation unit is configured to extract a green-channel image from the first image imaged from the first camera, and to calculate the first heart rate of the examinee based on the extracted green-channel image.

In one implementation of the first aspect, the first heart rate measuring module is disposed on the finger of the examinee, wherein the first heart rate measuring module is configured to irradiate light of a predetermined frequency to the finger of the examinee, to collect light beams reflected from or transmitted through the finger of the examinee, and to calculating the first heart rate of the examinee based on the collected light beams.

In one implementation of the first aspect, the second heart rate measuring module includes: a second camera for imaging the second location to obtain a second image; and a second heart rate calculation unit for calculating the second heart rate of the examinee based on the second image imaged from the second camera.

In one implementation of the first aspect, the second location corresponds to a facial portion of the examinee.

In one implementation of the first aspect, the second heart rate calculation unit is configured to extract a green-channel image from the second image imaged from the second camera, and to calculate the second heart rate of the examinee based on the extracted green-channel image.

In one implementation of the first aspect, the blood pressure estimation module includes: a time calculation unit for calculating a pulse transit time between the first and second locations based on the first and second heart rates provided by the first and second heart rate measuring modules; a velocity calculation unit for calculating a pulse wave velocity based on the pulse transit time calculated via the time calculation unit and, information on the body of the examinee; and a blood pressure estimation unit for estimating a blood pressure of the examinee based on the pulse wave velocity calculated via the velocity calculation unit.

In one implementation of the first aspect, the blood pressure measuring device further comprises a main body, wherein the first heart rate measuring module includes a first camera for imaging the first location, and the second heart rate measuring module includes a second camera for imaging the second location, wherein the first and second cameras are disposed on the main body.

In one implementation of the first aspect, the first location corresponds to a finger of the examinee and the second location corresponds to a facial portion of the examinee, wherein the second camera is disposed on a front face of the main body, and the first camera is disposed on a rear face of the main body such that the first and second locations are simultaneously imaged by the first and second cameras when the examinee grips the main body by a hand thereof.

In one implementation of the first aspect, the blood pressure measuring device further comprises: a display disposed on the front face of the main body for displaying thereon blood pressure data of the examinee calculated from the blood pressure estimation module; and a communication module disposed in the main body for transmitting the blood pressure data of the examinee calculated from the blood pressure estimation module to a management server and a terminal of a caregiver of the examinee.

In a second aspect of the present disclosure, there is provided a blood pressure measuring method comprising: measuring a first heart rate at a first location of an examinee's body; measuring a second heart rate at a second location of the examinee's body, wherein the first location is different from the second location, wherein a first spacing between the first location and a heart of the examinee is different from a second spacing between the second location and the heart of the examinee; and estimating a blood pressure of the examinee based on the first and second heart rates.

In one implementation of the second aspect, measuring the first heart rate includes: imaging the first location using a first camera to obtain a first image; and calculating the first heart rate of the examinee based on the obtained first image.

In one implementation of the second aspect, the first location corresponds to a finger of the examinee.

In one implementation of the second aspect, calculating the first heart rate of the examinee includes: extracting from the first image a first ROI (region of interest) image for the first heart rate calculation; extracting a first green-channel image from the first ROI the image to form a first analyzed image; and calculating the first heart rate of the examinee based on the first analyzed image.

In one implementation of the second aspect, the blood pressure measuring method further comprises filtering a noise from the first analyzed image, wherein the filtering occurs between extracting the first green-channel image and calculating the first heart rate.

In one implementation of the second aspect, measuring the second heart rate includes: imaging the second location using a second camera to obtain a second image; and calculating the second heart rate of the examinee based on the obtained second image.

In one implementation of the second aspect, the second location corresponds to a facial portion of the examinee.

In one implementation of the second aspect, calculating the second heart rate of the examinee includes: extracting from the second image a second ROI (region of interest) image for the second heart rate calculation; extracting a second green-channel image from the second ROI the image to form a second analyzed image; and calculating the second heart rate of the examinee based on the second analyzed image.

In one implementation of the second aspect, the blood pressure measuring method further comprises filtering a noise from the second analyzed image, wherein the filtering occurs between extracting the second green-channel image and calculating the second heart rate.

In one implementation of the second aspect, estimating the blood pressure includes: calculating a pulse transit time between the first and second locations based on the first and second heart rates; calculating a pulse wave velocity based on the pulse transit time, and information on the body of the examinee; and estimating a blood pressure of the examinee based on the pulse wave velocity.

In one implementation of the second aspect, the blood pressure measuring method further comprises: setting a main body having first and second cameras disposed on rear and front faces thereof respectively, wherein setting the main body includes gripping the main body by a user's hand such that a finger of the examinee as the first location is adjacent to the first camera and the second camera faces a facial portion of the examinee as the second location, thereby to allow the first and second cameras to image the finger and facial portion, wherein measuring the first heart rate includes: imaging the finger using the first camera to obtain a first image; and calculating the first heart rate of the examinee based on the obtained first image, wherein measuring the second heart rate includes: imaging the facial portion using the second camera to obtain a second image; and calculating the second heart rate of the examinee based on the obtained second image.

According to a third aspect of the present disclosure, there is provided a real-time blood flow change measurement method. The method includes: a first operation for imaging a facial image in real time using a camera module; a second operation for calculating a skin color change amount from the facial image; and a third operation for measuring a blood flow change signal and a blood flow change image from the calculated skin color change amount.

In one implementation of the third aspect, the second operation for calculating a skin color change amount from the facial image may include modeling the facial image using a Lambert Beer rule, wherein modeling the facial image using the Lambert Beer rule includes dividing the facial image into a portion resulting from melanin, a portion resulting from hemoglobin, and a residual portion.

In one implementation of the third aspect, the method further includes, prior to the second operation for calculating the skin color change amount, an operation for down-sampling the facial image, and an operation for carrying out HSV correction thereto. In the second operation for calculating the skin color change amount after the HSV correction is performed, an adaptive filtering may be recursively applied to minimize a motion artifact in the skin color change amount. In this connection, the adaptive filtering may be any of, but is not limited to, an LMS (Least Mean Square) filter, an NLMS (Normalized LMS) filter, and an RLS (Recursive Least Square) filter.

In one implementation of the third aspect, the method further includes an operation of real-time estimation of a change in heart rate from the blood flow change signal and the blood flow change image.

In one implementation of the third aspect, the third operation for measuring the blood flow change signal and the blood flow change image from the calculated skin color change amount includes: an operation for deriving a hemoglobin change amount from the calculated skin color change amount; an operation for filtering of the hemoglobin change amount to obtain a filtered result; an operation for up-sampling the filtered result to measure the blood flow change image; and an operation for averaging the filtered result to measure the blood flow change signal.

According to a fourth aspect of the present disclosure, there is provided a computer-readable storage medium having a computer program stored therein, wherein when the program is executed by the computer, the program allows the computer to perform a real-time blood flow change measurement method, wherein the computer program comprises instructions for imaging a facial image using a camera module in real-time; instructions for calculating a skin color change amount from the facial image; and instructions for measuring a blood flow change signal and a blood flow change image from the calculated skin color change amount.

According to a fifth aspect of the present disclosure, there is provided a real-time blood flow change measurement device. The device includes: a camera module for imaging a facial image in real time; and an image processing module for calculating a skin color change amount from the facial image, and for measuring a blood flow change signal and a blood flow change image from the calculated skin color change amount.

In one implementation of the fifth aspect, the image processing module for calculating the skin color change amount from the facial image is configured to model the facial image using a Lambert Beer rule, wherein the image processing module for modeling the facial image using the Lambert Beer rule is further configured to divide the facial image into a portion resulting from melanin, a portion resulting from hemoglobin, and a residual portion.

In one implementation of the fifth aspect, in order to reduce the calculation amount and increase the accuracy of the calculation in calculating the amount of skin color change, the image processing module includes: a down-sampling unit for down-sampling the facial image; a correction unit for carrying out HSV correction of the down-sampled facial image; and an adaptive filtering unit for minimizing motion artifacts in the skin color change amount by updating a weight.

In one implementation of the fifth aspect, the image processing module comprises: an up-sampling unit for deriving the blood flow change image after the facial image is subjected to the adaptive filtering; and an averaging unit for deriving the blood flow change signal after the facial image is subjected to the adaptive filtering.

As for the blood pressure measuring device according to the present disclosure, constructed as described above, and the blood pressure measuring method using the device as described above, the first image of the facial portion of the examinee and the second image of the finger different in spacing from the heart from the facial portion are imaged and thus the blood pressure is calculated using the first and second images. Therefore, the device is relatively small in volume, thus easy to carry, and simple to use, so that the non-specialist can easily measure the blood pressure.

According to the present disclosure, the skin color change amount, blood flow change image and signal, and heart rate change amount may be measured in real time from the image obtained by the camera module.

Particularly, according to the method or algorithm proposed in the present disclosure, since the amount of computation required to measure the blood flow change to the facial portion in real-time is small and, thud, the load on the hardware to execute the computation is low, the device of the present disclosure can be implemented as a user device such as a smart phone capable of data communication and capable of real-time photo imaging.

Those skilled in the art will appreciate that effects of the present disclosure are not limited to the above-mentioned effects.

### BRIEF DESCRIPTION OF THE DRAWINGS

The accompanying drawings, which are incorporated in and form a part of this specification and in which like numerals depict like elements, illustrate embodiments of the present disclosure and, together with the description, serve to explain the principles of the disclosure.

FIG. 1 and FIG. 2 are perspective views of a blood pressure measuring device according to one embodiment of the present disclosure.

FIG. 3 is a block diagram of the blood pressure measuring device according to one embodiment of the present disclosure.

FIG. 4 is a flowchart illustrating a method of measuring a blood pressure using the blood pressure measuring device according to one embodiment of the present disclosure.

FIG. 5 is a graph of a heart rate calculated from an image of a finger of an examinee using the blood pressure measuring device according to one embodiment of the present disclosure.

FIG. 6 is a graph of a heart rate calculated from an image of a facial portion of an examinee using the blood pressure measuring device according to one embodiment of the present disclosure.

FIG. 7 shows a comparison between a blood pressure of an examinee measured according to the blood pressure measuring method using the blood pressure measuring device 100 according to an embodiment of the present disclosure and a blood pressure of the examinee measured using a conventional oscillometric blood pressure measuring device.

FIG. 8 is a schematic flowchart of a method for measuring real-time blood flow change according to another embodiment of the present disclosure.

FIG. 9 is a block diagram of a device for measuring real-time blood flow change according to another embodiment of the present disclosure.

FIG. 10 is a schematic diagram of an algorithm in which a real-time blood flow change measurement method, device, and computer-readable storage medium is implemented, according to another embodiment of the present disclosure.

FIG. 11 are images for illustrating an experimental example of a method of measuring real-time blood flow change according to another embodiment of the present disclosure.

FIG. 12 shows a graph comparing experimental results of heart rate estimation according to types of the adaptive filters used in the real-time blood flow measurement method according to the present disclosure

### DETAILED DESCRIPTIONS

For simplicity and clarity of illustration, elements in the figures are not necessarily drawn to scale. The same reference numbers in different figures denote the same or similar elements, and as such perform similar functionality. Also, descriptions and details of well-known steps and elements are omitted for simplicity of the description. Furthermore, in the following detailed description of the present disclosure, numerous specific details are set forth in order to provide a thorough understanding of the present disclosure. However, it will be understood that the present disclosure may be practiced without these specific details. In other instances, well-known methods, procedures, components, and circuits have not been described in detail so as not to unnecessarily obscure aspects of the present disclosure.

Examples of various embodiments are illustrated and described further below. It will be understood that the description herein is not intended to limit the claims to the specific embodiments described. On the contrary, it is intended to cover alternatives, modifications, and equivalents as may be included within the spirit and scope of the present disclosure as defined by the appended claims.

It will be understood that, although the terms “first”, “second”, “third”, and so on may be used herein to describe various elements, components, regions, layers and/or sections, these elements, components, regions, layers and/or sections should not be limited by these terms. These terms are used to distinguish one element, component, region, layer or section from another element, component, region, layer or section. Thus, a first element, component, region, layer or section described below could be termed a second element, component, region, layer or section, without departing from the spirit and scope of the present disclosure.

It will be understood that when an element or layer is referred to as being “connected to”, or “coupled to” another element or layer, it can be directly on, connected to, or coupled to the other element or layer, or one or more intervening elements or layers may be present. In addition, it will also be understood that when an element or layer is referred to as being “between” two elements or layers, it can be the only element or layer between the two elements or layers, or one or more intervening elements or layers may also be present.

Spatially relative terms, such as “beneath,” “below,” “lower,” “under,” “above,” “upper,” and the like, may be used herein for ease of explanation to describe one element or feature's relationship to another element s or feature s as illustrated in the figures. It will be understood that the spatially relative terms are intended to encompass different orientations of the device in use or in operation, in addition to the orientation depicted in the figures. For example, if the device in the figures is turned over, elements described as “below” or “beneath” or “under” other elements or features would then be oriented “above” the other elements or features. Thus, the example terms “below” and “under” can encompass both an orientation of above and below. The device may be otherwise oriented for example, rotated 90 degrees or at other orientations, and the spatially relative descriptors used herein should be interpreted accordingly.

The terminology used herein is for the purpose of describing particular embodiments only and is not intended to be limiting of the present disclosure. As used herein, the singular forms “a” and “an” are intended to include the plural forms as well, unless the context clearly indicates otherwise. It will be further understood that the terms “comprises”, “comprising”, “includes”, and “including” when used in this specification, specify the presence of the stated features, integers, operations, elements, and/or components, but do not preclude the presence or addition of one or more other features, integers, operations, elements, components, and/or portions thereof. As used herein, the term “and/or” includes any and all combinations of one or more of the associated listed items. Expression such as “at least one of” when preceding a list of elements may modify the entire list of elements and may not modify the individual elements of the list.

Unless otherwise defined, all terms including technical and scientific terms used herein have the same meaning as commonly understood by one of ordinary skill in the art to which this inventive concept belongs. It will be further understood that terms, such as those defined in commonly used dictionaries, should be interpreted as having a meaning that is consistent with their meaning in the context of the relevant art and will not be interpreted in an idealized or overly formal sense unless expressly so defined herein.

In the following description, numerous specific details are set forth in order to provide a thorough understanding of the present disclosure. The present disclosure may be practiced without some or all of these specific details. In other instances, well-known process structures and/or processes have not been described in detail in order not to unnecessarily obscure the present disclosure.

As used herein, the term “substantially,” “about,” and similar terms are used as terms of approximation and not as terms of degree, and are intended to account for the inherent deviations in measured or calculated values that would be recognized by those of ordinary skill in the art. Further, the use of “may” when describing embodiments of the present disclosure refers to “one or more embodiments of the present disclosure.”

### First Embodiment

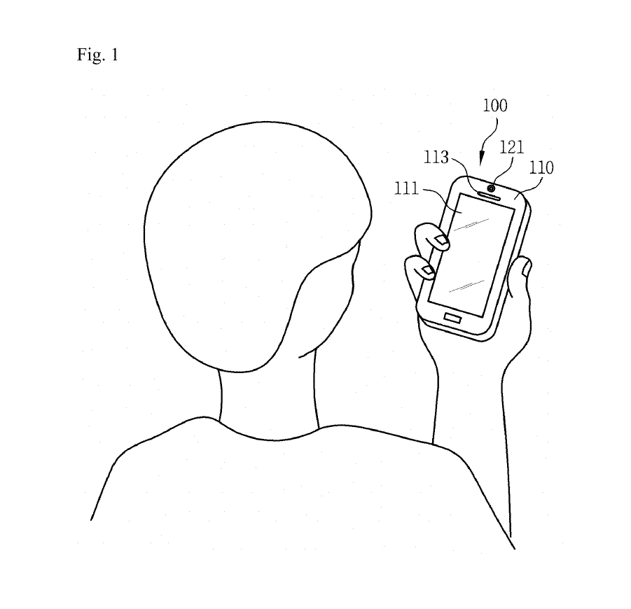

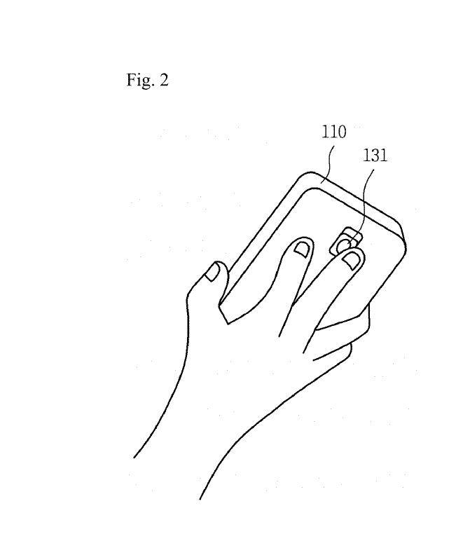

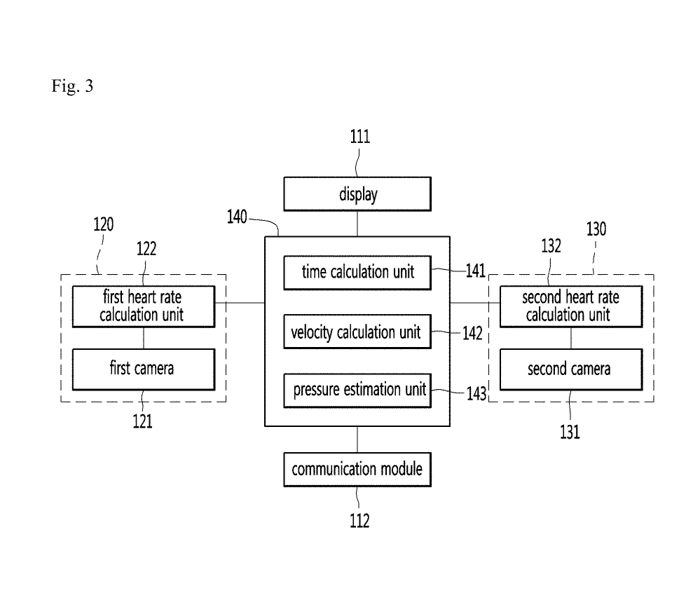

FIG. 1 to FIG. 3 show a blood pressure measuring device 100 according to one embodiment of the present disclosure.

Referring to the drawings, the blood pressure measuring device 100 includes a main body 110, a first heart rate measuring module 120 for measuring a first heart rate at a first location of a body of an examinee holding the main body 110, a second heart rate measuring module 130 for measuring a second heart rate at a second location of the body of the examinee, and a blood pressure estimation module 140 for estimating a blood pressure of the examinee based on the first and second heart rates measured by the first and second heart rate measuring modules 120 and 130 respectively. The first heart rate measuring module 120, the second heart rate measuring module 130 and the blood pressure estimation module 140 are installed in the main body 110.

The main body 110 has a rectangular structure so that the examinee can grasp easily. The main body 110 has a display 111 installed on its front side. Thus, the blood pressure data of the examinee estimated from the blood pressure estimation module may be displayed on the display 111. Further, in the main body 110, there are a communication module 112 to realize a telephone function with a terminal of another user and to send data including the blood pressure data of the examinee as calculated from the blood pressure estimation module to a management server and a terminal of a sponsor of the examinee, a microphone (not shown) for receiving the voice of the examinee, a loudspeaker 113 for outputting voice information, and a central processing unit to control the display 111, the speaker 113, the microphone, and the communication module 112. The main body 110 may be implemented as a conventional smart phone capable of communicating with other persons and processing input information. In the main body 110, a memory (not shown) in which a plurality of pieces of information are stored is installed. In the memory, there is stored body information of the examinee measured via the body measuring device, for example, a height of the examinee, and spacings from the heart of the examinee to the examinee's finger and face. In this connection, the examinee may directly store his/her body information into the memory in the main body 110 when measuring the blood pressure.

The first heart rate measuring module 120 includes a first camera 121 for imaging the first location, and a first heart rate calculation unit 122 for calculating the first heart rate of the examinee based on a first image captured from the first camera 121. In this connection, the first location may be the finger of the examinee.

The first camera 121 is installed at a top of the rear portion of the main body 110 so as to image the finger when the examinee grasps the main body 110. The first camera 121 captures an image of the finger when the examinee's finger is adjacent thereto.

The first heart rate calculation unit 122 is provided inside the main body 110 and is configured to extract a green-channel image from the first image captured from the first camera 121 and calculate the first heart rate of the examinee based on the green-channel image. In this connection, the first heart rate calculation unit 122 extracts a region of interest (ROI) associated with the heart rate from the image captured from the first camera 121 and extracts the green-channel image from the extracted ROI. Further, the first heart rate calculation unit 122 extracts the heart rate by removing noises from the extracted green-channel image. In this regard, a Kalman filter or an adaptive filter, etc. may be used for removing the noises.

The first heart rate calculation unit 122 may be a separate unit or may implemented as the central processing unit in the main body 110. At the latter case, the central processing unit in the main body 110 is configured to calculate the heart rate based on the image captured from the first camera 121. Further, the first heart rate calculation unit 122 may include an image processing module as described later.

Alternatively, instead of measuring the heart rate using a finger image taken by the first camera 121, the first heart rate measuring module 120 may employ alternative means, although not shown in the figure. For example, a light sensor is mounted on the finger of the examinee, the light of a predetermined wavelength is irradiated from the light sensor to the finger of the examinee, the light reflected from or transmitting through the finger of the examinee is collected, and, thus, the heart rate may be calculated based on the collected light. Alternatively, a metal electrode sensor may be installed on the finger and collect an electrical signal from the examinee's finger and measure the heart rate based on the electrical signal. In this connection, the optical sensor measures the heart rate using the finger of the examinee, and is a generally used electrocardiogram measuring instrument, and thus a detailed description thereof will be omitted.

Further, the first location is not limited to the examinee's finger. The first location may be any portions of the body of the examinee except the second location of the examinee. The first heart rate measuring module 120 may include the camera for capturing images and/or means for measuring the heart rate as conventionally used, such as an optical sensor or an oscillometric measurement device.

The second heart rate measuring module 130 includes a second camera 131 for imaging the second location, and a second heart rate calculation unit 132 for calculating the second heart rate of the examinee based on the second image captured from the second camera 131. In this connection, the second location may be the facial portion of the examinee.

The second camera 131 is installed at a top of a front portion of the main body 110 so as to image the facial portion when the examinee grasps the main body 110.

The second heart rate calculation unit 132 is provided inside the main body 110 and is configured to extract a green-channel image from the second image captured from the second camera 131 and calculate the second heart rate of the examinee based on the green-channel image. In this connection, the second heart rate calculation unit 132 extracts a region of interest (ROI) associated with the heart rate from the image captured from the second camera 131 and extracts the green-channel image from the extracted ROI. Further, the second heart rate calculation unit 132 extracts the heart rate by removing noises from the extracted green-channel image. In this regard, a Kalman filter or an adaptive filter, etc. may be used for removing the noises.

The second heart rate calculation unit 132 may be a separate unit or may implemented as the central processing unit in the main body 110. At the latter case, the central processing unit in the main body 110 is configured to calculate the heart rate based on the image captured from the second camera 131. Further, the second heart rate calculation unit 132 may include an image processing module as described later.

In one embodiment, the first location may be a finger of the examinee and the second location may be a facial portion of the examinee, but the first and second locations are not limited thereto. In general, the first and second locations may correspond to any two different portions of the body of the examinee having different spacings from the heart respectively. The installation positions of the first and second cameras 121 and 131 in the main body 110 may be changed or may be installed separately from the main body 110, based on the set first and second locations.

The blood pressure estimation module 140 include a time calculation unit 141 for calculating a pulse transit time between the first and second locations based on the first and second heart rates provided from the first and second heart rate measuring modules 120 and 130, a velocity calculation unit 142 for calculating a pulse wave velocity based on the pulse transit time calculated by the time calculation unit 141 and the body information of the examinee, and a blood pressure estimation unit 143 for estimating the blood pressure of the examinee based on the pulse wave velocity calculated by the velocity calculation unit 142.

The time calculation unit 141 detects mutually-corresponding first and second peak points of the first heart rate provided from the first heart rate measuring module 120 and the second heart rate provided from the second heart rate measuring module 130, and calculates the pulse transit time based on a difference between measurement times of the first and second peak points calculated as described above.

The velocity calculation unit 142 calculates the pulse wave velocity based on the body information of the user stored in the memory of the main body 110, that is, the height of the examinee, and the spacings between the heart and the finger and the facial portion of the examinee, and the pulse transit time as calculated.

The blood pressure estimation unit 143 estimates the blood pressure of the examinee based on the pulse wave velocity calculated by the velocity calculation unit 142. The blood pressure estimation unit 143 may use a regression model between the pulse wave velocity and the blood pressure to improve the accuracy of the blood pressure estimation. In this connection, the operator stores the relationship data between the pulse wave velocity and the blood pressure value of the examinee obtained through a plurality of experiments into the memory of the main body 110. The blood pressure estimation unit 143 may more accurately estimate the blood pressure value of the examinee based on the relationship data.

The blood pressure estimation module 140 may be separately provided in the main body 110. Alternatively, the blood pressure estimation module 140 may be implemented by the central processing unit of the main body 110. In the latter case, the central processing unit is configured to estimate the blood pressure of the examinee based on the first and second heart rates measured via the first and second heart rate measuring modules 120 and 130 respectively.

A blood pressure measuring method using the blood pressure measuring device 100 according to the present disclosure, as constructed as described above will be described in detail below.

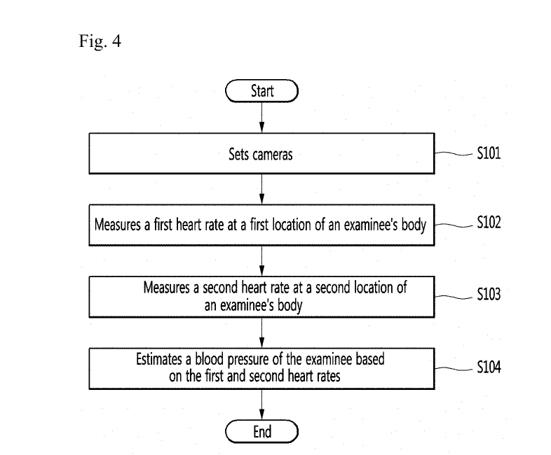

FIG. 4 is a flowchart illustrating a method of measuring the blood pressure according to an exemplary embodiment of the present disclosure. Referring to the drawing, the blood pressure measurement method includes a setting operation S101, a first heart rate measurement operation S102, a second heart rate measurement operation S103, and a blood pressure estimation operation S104.

In the setting operation S101, the main body 110 having the first camera 121 and the second camera 131 installed on the rear and front portions of the main body 110, respectively, is set. In this connection, the examinee adjoins a finger of the examinee to the first camera 121 so that the first camera 121 photographs the finger of the examinee, which is the first location. Further, the examinee grasps the main body 110 such that the second camera 131 faces the facial portion of the examinee so that the facial portion of the examinee, which is the second location, is photographed by the second camera 131.

In the first heart rate measurement operation S102, the first heart rate is measured on the finger, which is the first location of the body of the examinee. The first heart rate measurement operation S102 includes a first imaging operation and a first heart rate calculation operation.

The first imaging operation involves imaging the image of the first location of the examinee using the first camera 121. The examinee positions the finger adjacent to the first camera 121 and then manipulates the first camera 121 to be activated.

The first heart rate calculation operation includes an operation to calculate the heart rate based on the image imaged through the first imaging operation. The first heart rate calculation operation includes a first image extraction operation, a first preparation operation, a first filtering operation, and a first calculation operation.

The first image extraction operation includes an operation of extracting a ROI image for the heart rate calculation from the image imaged through the first imaging operation. When the examinee's finger image is imaged by the first camera 121, the first heart rate calculation unit 122 extracts the region of interest (ROI) associated with the heart rate from the image imaged from the first camera 121.

The first preparation operation includes an operation of extracting a green-channel image from the ROI image extracted through the first image extraction operation to form a first analyzed image. The first heart rate calculation unit 122 extracts the green-channel image from the extracted ROI to form the first analyzed image.

The first filtering operation includes an operation of removing noise from the first analyzed image between the first preparation operation and the first calculation operation. In this connection, the first heart rate calculation unit 122 removes noise from the first analyzed image using an image filter such as a Kalman filter or an adaptive filter.

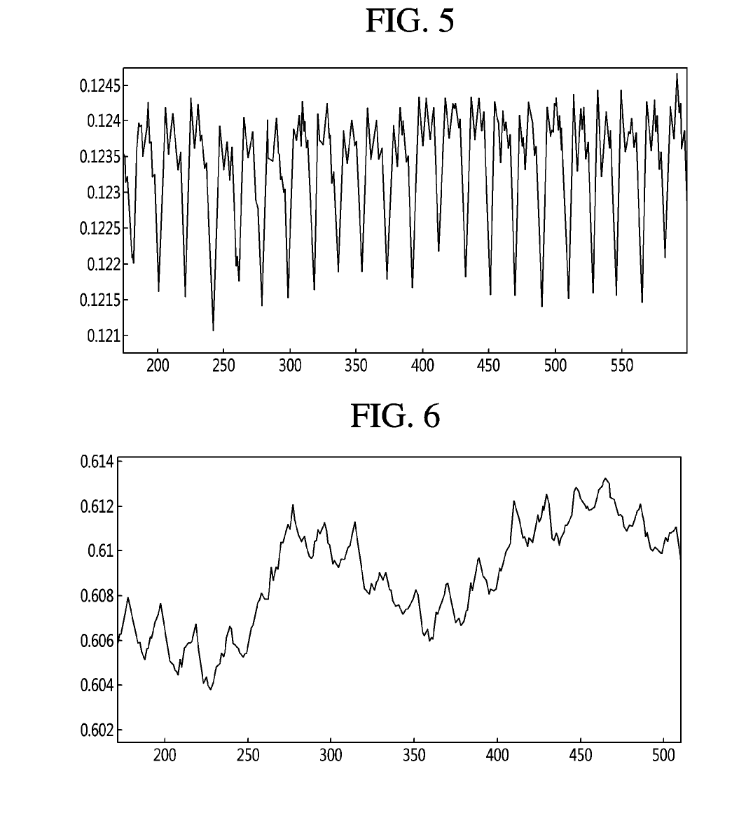

The first calculation operation includes an operation for calculating the heart rate of the examinee based on the first analyzed image. FIG. 5 is a graph of the heart rate calculated from the finger image of the examinee as actually performed. In this graph, an x-axis is a measurement time and a y-axis is a heart rate.

The second heart rate measurement operation S103 includes an operation to measure the second heart rate at the facial portion, which is the second location of the body of the examinee. The second heart rate measurement operation S103 includes a second imaging operation and a second heart rate calculation operation.

The second imaging operation involves imaging the image of the second location of the examinee using the second camera 131. The examinee positions the facial portion adjacent to the second camera 131 and then manipulates the second camera 131 to be activated.

The second heart rate calculation operation includes an operation to calculate the second heart rate based on the image imaged through the second imaging operation. This second heart rate calculation operation includes a second image extraction operation, a second preparation operation, a second filtering operation, and a second calculation operation.

The second image extraction operation includes an operation to extract the ROI image for the heart rate calculation from the image imaged through the second imaging operation. When the image of the facial portion of the examinee is imaged by the second camera 131, the second heart rate calculation unit 132 extracts the ROI (Region of interest) associated with the heart rate from the image imaged from the second camera 131.

The second preparation operation includes an operation of extracting a green-channel image from the ROI image extracted through the second image extracting operation to form a second analyzed image. The second heart rate calculation unit 132 extracts the green-channel image from the extracted ROI to form a second analyzed image.

The second filtering operation includes an operation of removing noise from the second analyzed image between the second preparation operation and the second calculation operation. In this connection, the second heart rate calculation unit 132 uses the image filter such as a Kalman filter or an adaptive filter to remove noise from the second analyzed image.

The second calculation operation includes an operation of calculating the second heart rate of the examinee based on the second analyzed image. FIG. 6 is a graph of the heart rate calculated from the image of the facial portion of the examinee as actually performed. In this graph, an x-axis is a measurement time and a y-axis is a heart rate.

In one embodiment, it is preferable that the first and second heart rate calculation operations are performed simultaneously.

The blood pressure estimation operation S104 includes an operation for estimating the blood pressure of the examinee based on the first and second heart rates at the first and second locations measured by the first and second heart rate calculation operations. The blood pressure estimation operation S104 includes a time calculation operation, a velocity calculation operation, and a blood pressure calculation operation.

The time calculation operation calculates a pulse transit time between the first and second locations based on the first and second heart rates at the first and second locations measured by the first and second heart rate measurement operations S102 and S103. The time calculation unit 141 in the blood pressure estimation module 140 is configured to receive the first heart rate from the first heart rate measuring module 120 and the second heart rate from the second heart rate measuring module 130, to detect mutually-corresponding first and second peak points of the first and second heart rates, to calculate a difference between measurement times of the mutually-corresponding first and second peak points, and to calculate a pulse transit time based on the calculated measurement time difference.

The velocity calculation operation includes an operation to calculate the pulse wave velocity based on the pulse transit time calculated from the time calculation operation and the body information of the examinee. The velocity calculation unit 142 in the blood pressure estimation module 140 calculates the pulse wave velocity based on the calculated pulse transit time by the time calculation unit 141 and the body information.

The blood pressure calculation operation includes an operation of estimating the blood pressure of the examinee using the pulse wave velocity calculated through the velocity calculation operation. The blood pressure estimation unit 143 in the blood pressure estimation module 140 estimates the blood pressure of the examinee based on the calculated pulse wave velocity through the velocity calculation unit 142. In this connection, the operator may store the relationship data between the examinee's pulse wave velocity and the blood pressure value as obtained through multiple experiments into the memory of the main body 110. Thereafter, the blood pressure estimation unit 143 may more accurately estimate the blood pressure value of the examinee based on the relationship data.

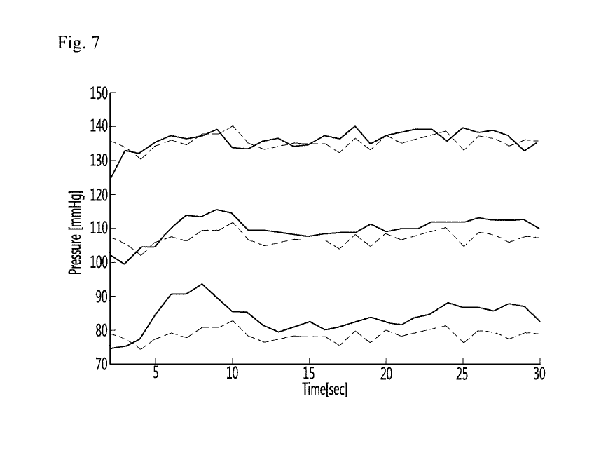

FIG. 7 shows a comparison between a blood pressure of an examinee measured according to the blood pressure measuring method using the blood pressure measuring device 100 according to an embodiment of the present disclosure and a blood pressure of the examinee measured using a conventional oscillometric blood pressure measuring device. In this connection, the x-axis refer to the measurement time and the y-axis refers to the blood pressure value. The blue graph refers to the blood pressure of the examinee measured according to the blood pressure measurement method using the blood pressure measuring device 100 according to the present disclosure. The red graph refers to the blood pressure of the examinee measured using the conventional oscillometric blood pressure measuring device. The blue graph and the red graph at the bottom in the graph refer to the blood pressure values at the diastole of the heart, while the blue graph and the red graph at the top in the graph refer to the blood pressure values at the heart's systole. The middle blue graph and the red graph refer to the blood pressure values averaged over the measurement time. Referring to FIG. 7, it may be confirmed that the blood pressure value of the examinee measured according to the blood pressure measuring method using the blood pressure measuring device 100 according to the present disclosure is substantially equal to that as measured by using the conventional oscillometric blood pressure measuring device.

As for the blood pressure measuring device 100 according to the present disclosure, constructed as described above, and the blood pressure measuring method using the device 100 as described above, the first image of the facial portion of the examinee and the second image of the finger different in spacing from the heart from the facial portion are imaged and thus the blood pressure is calculated using the first and second images. Therefore, the device 100 is relatively small in volume, thus easy to carry, and simple to use, so that the non-specialist can easily measure the blood pressure.

### Second Embodiment

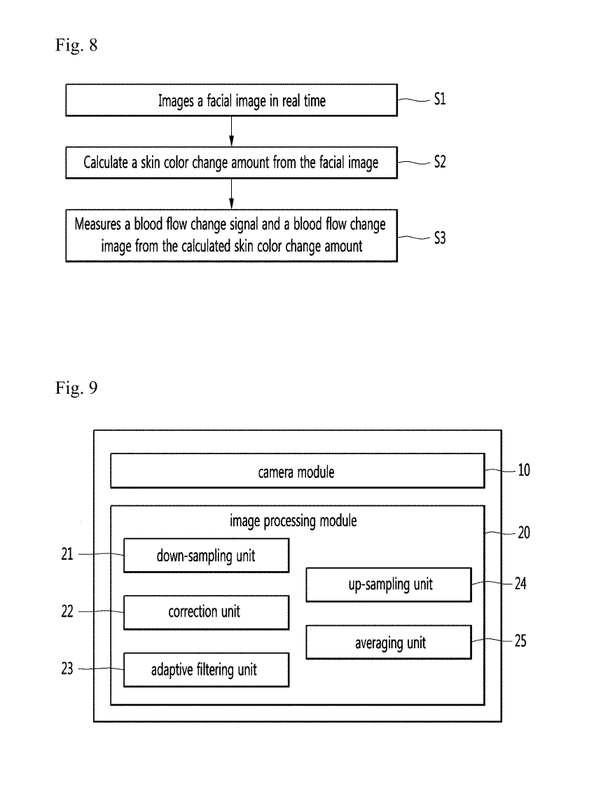

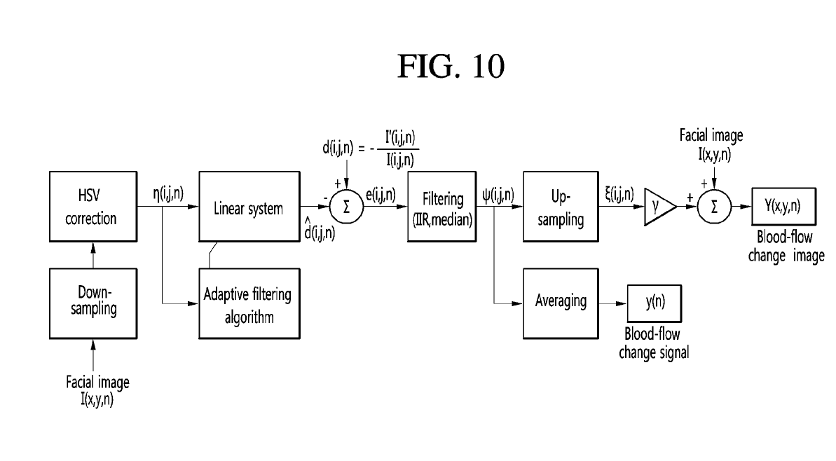

FIG. 8 is a schematic flowchart of a method for measuring real-time blood flow change according to another embodiment of the present disclosure. FIG. 9 is a block diagram of a device for measuring real-time blood flow change according to another embodiment of the present disclosure. FIG. 10 is a schematic diagram of an algorithm in which a real-time blood flow change measurement method, device, and computer-readable storage medium is implemented, according to another embodiment of the present disclosure. With respect to each subject performing each operation of FIG. 8, reference is made to FIG. 9, which is a block diagram of the real-time blood flow change measuring device, and is made to FIG. 10, which is a schematic diagram of the algorithm.

In a first operation in FIG. 8, a facial image is imaged in real time through a camera module (10 in FIG. 9). In this connection, the camera module 10 may be a web cam usually provided in a user's computer, but the present disclosure is not limited thereto. Any camera module may be implemented as the camera module 10 if image processing is possible locally/remotely and imaging is possible in real-time by the same.

In the second operation in FIG. 8, the amount of skin color change is calculated from the facial image imaged in real-time by an image processing module 20. In a third operation thereof, a blood flow change amount and a blood flow change image may be measured from the calculated skin color change amount. These second and third operations may be performed by the image processing module (20 of FIG. 10). In the second operation, in calculating the amount of skin color change, according to the present disclosure, a modeling may be performed with respect to the facial image imaged/input in real-time using a Lambert beer rule to distinguish between a factor derived from melanin and a factor derived from hemoglobin.

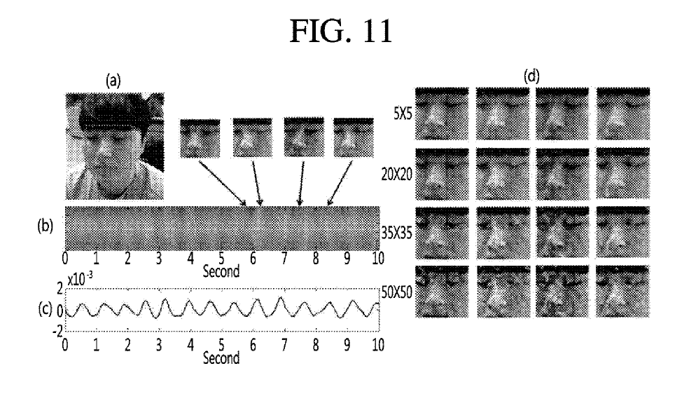

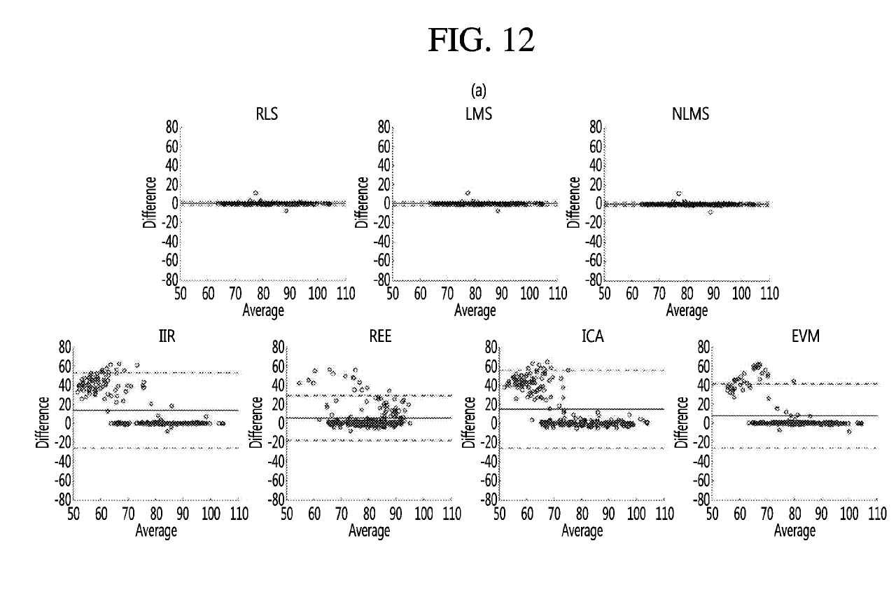

In this connection, in order to reduce the amount of calculation accompanying the modeling by applying the Lambert Beer rule, a down-sampling unit (21 in FIG. 9) down-samples the facial image to an arbitrary frequency. For example, if the image imaged in real-time is 200 x 200, the image may be down-sampled to 5×5, 20×20, 35×35, 50×50, etc., depending on the sampling frequency. This embodiment is illustrated in FIG. 11. For the facial image imaged as shown in FIG. 11A, examples of down-sampled images are shown as FIG. 11D. Through the down-sampling process, the amount of computation may be reduced, and, further, ultimately, the amount of change in the blood flow may be expressed smoothly.

After down-sampling, HSV correction is performed by the correction unit 22. This allows to equalize the intensity and thus reduces motion artifact factor. This is further described when describing the modeling in accordance with Lambert Beer's law.

Again, the modeling according to the Lambert-Beer law will be described. When an arbitrary light reaches the skin, the degree of light transmission may vary depending on the frequency band. In order to calculate the amount of change in skin color, modeling is performed with dividing various factors determining skin color into (i) a factor attributable to melanin, (ii) a factor attributable to hemoglobin, and (iii) a residual portion. Thus, using the Lambert Beer rule, the facial image may be modeled as Equation 1 below:

A(λ,n)=vₘ(λ, n)cₘ+vₕ(λ,n)cₕ+A₀(λ,n) (1)
λ: frequency,
n: discrete time index
cₘ, cₕ: pigment concentration
vₘ: extinction coefficient of melanin
vₕ: Extinction coefficient of hemoglobin
A: absorption rate
A₀: residual

In Equation 1, the absorption rate A may be expressed by an intensity E of incident light and an intensity L of transmitted light as A=−log(L/E). Thus, a following equation 2 may be derived by applying A=−log(L/E).

L(λ,n)=Eexp{−(vₘ(λ,n)cₘ+vₕ(λ,n)cₕ+A₀(λ,n))} (2)

The above equation 2 may be integrated using all frequency bands and x and y as coordinates of entire pixels of the facial image, such that Equation 3 may be derived as follows:

i(x,y,n)=G∫L(x,y,λ,n)S(λ)dλ (3)

In this regard, assuming that the spectral response function S is a Kronecker delta function, Equation 2 is applied to L (x, y, λ, n) in Equation 3, resulting in i (x, y, n) as expressed as follows:

i(x,y,n)=GEexp{−(vₘ)cₘ+vₕ(x,y,n)cₕ+A₀(x,y,n))} (4)

When the equation 4 is transformed into a natural logarithmic form, it is assumed that G and E are arbitrary constants, and the amount of change in skin color is represented by

$$- \frac{{i}^{′} ( x , y , n )}{i ( x , y , n )} ,$$

the amount of change in skin color may be represented by a following equation (5) including (i) a factor due to melanin, (ii) a factor due to hemoglobin, and (iii) a residual portion:

$$- \frac{{i}^{′} ( x , y , n )}{i ( x , y , n )} = {v}_{m}^{′} ( x , y , n ) {c}_{m} + {v}_{h}^{′} ( x , y , n ) {c}_{h} + {A}_{0}^{′} ( x , y , n ) . \qquad ( 5 )$$

Since the amount of change in the factor due to hemoglobin is extremely small compared with the amount of change in the factor due to melanin and the amount of change in the remaining portion, in the relational expression for the amount of change in skin color, as expressed by the equation 5, the equation 5 may be approximated to a following equation 6:

$$- \frac{{i}^{′} ( x , y , n )}{i ( x , y , n )} \approx {v}_{m}^{′} ( x , y , n ) {c}_{m} + {A}_{0}^{′} ( x , y , n ) \{ \begin{matrix}{v}_{\text{?}}^{′} ( x , y , n ) >> {v}_{\text{?}}^{′} ( x , y , n ) \\ {A}_{\text{?}}^{′} ( x , y , n ) >> {v}_{\text{?}}^{′} ( x , y , n )\end{matrix} . \\ \text{?} \text{indicates text missing or illegible when filed} \qquad ( 6 )$$

Thus, with respect to the modeling of the amount of skin color change expressed by the factor resulting from melanin and by the remaining portion, a process for eliminating motion artifacts due to user motion is now described.

Conventional adaptive filters are known to remove noise. By applying this adaptive filter (for example, an adaptive filter portion 25 in FIG. 9) to the model, it is possible to reduce the motion artifact associated with the melanin-attributable factor and the residual portion. In accordance with the present disclosure, the adaptive filter may preferably be a least mean square (LMS) filter, a normalized least mean square (NLMS) filter, or a recursive least square (RLS) filter, but the present disclosure is not limited thereto.

In other words, by applying the adaptive filter, optimal blood flow changes with minimal effect of motion artifact on skin color change amount can be obtained. By applying such an adaptive filter to all pixels, the change in blood flow can be estimated in real time.

In this connection, if an adaptive filter algorithm is applied to all the pixels of the facial image, the computational load increases and thus the load on the hardware increases accordingly. Thus, the down-sampling needs to be performed before applying the adaptive filter algorithm.

Down-sampling (21 in FIG. 9) with respect to the input facial image to an arbitrary frequency is performed. Subsequently, a HSV correction process (22 in FIG. 9) is performed to even out the intensity, and the motion artifact is reduced. By applying a linear system and the adaptive filter to the result thus obtained, the amount of change in skin color may be derived.

In this way, after the process of minimizing the motion artifact with respect to the amount of skin color change, and a filtering process (e.g., IIR, median, etc.) of the calculated amount of skin color change (as in the third operation shown in FIG. 8), the blood flow change image may be obtained through an up-sampling process (via an up-sampling unit 24 in FIG. 9). Then, a blood flow change signal may be calculated through an averaging process (via an averaging unit 25 in FIG. 9) of the filtered skin color change amount.

Further, from the blood flow change signal and the blood flow change image calculated by the third operation shown in FIG. 8, the change of the heart rate may be estimated in real time. In this regard, FIG. 12 shows a graph comparing experimental results of heart rate estimation according to types of the adaptive filters used in the real-time blood flow measurement method according to the present disclosure. Referring to FIG. 12, distributions of heart rate estimations resulting from when the adaptive filters according to RLS, LMS, and NLMS as in an upper part of FIG. 12 are used are substantially equal to those using conventional research methods (HR, REE, ICA, EVM) as in a lower portion of FIG. 12. Therefore, it may be seen that the accuracy of the technique proposed by the present disclosure is improved.

It is to be understood that the example modules, units, logic blocks, operations, or combinations thereof in the embodiments described herein may be implemented as electronic hardware, that is, digital design designed by coding, software, i.e., various types of applications including program instructions, or a combination thereof. Whether or not the exemplary module, unit, logic block, operation, or combination thereof is implemented in hardware and/or software may depend on design constraints imposed on the user terminal.

In some embodiments, the example modules, units, logic blocks, operations, or combinations thereof in the embodiments described herein may be stored in memory as computer program instructions. Such computer program instructions may be associated with a digital signal processor to perform the methods described herein. The examples of connections between the components specified with reference to the drawings attached hereto are merely exemplary, and at least some of the components may be omitted, and conversely, additional components may be further included.

According to an embodiment of the present disclosure, the above-mentioned method can be embodied as processor readable codes on a non-transitory processor readable recording medium having a program thereon. Examples of the processor readable recording medium include ROM, RAM, CD-ROM, magnetic tape, floppy disk, and an optical data storage device and also include carrier waves e.g., transmission through the Internet .

Although the disclosure has been described with reference to the exemplary embodiments, the present disclosure is not limited thereto and those skilled in the art will appreciate that various modifications and variations can be made in the present disclosure without departing from the spirit or scope of the disclosure. For example, those skilled in the art may modify the components of the embodiments. Differences related to such modifications and applications are interpreted as being within the scope of the present disclosure described in the appended claims.

What is claimed is:

1. A blood pressure measuring device comprising:
a first heart rate measuring module configured to measure a first heart rate at a first location of an examinee's body;
a second heart rate measuring module configured to measure a second heart rate at a second location of the examinee's body, wherein the first location is different from the second location, wherein a first spacing between the first location and a heart of the examinee is different from a second spacing between the second location and the heart of the examinee; and
a blood pressure estimation module configured to estimate a blood pressure of the examinee based on the first and second heart rates measured by the first and second heart rate measuring modules.

2. The blood pressure measuring device of claim 1, wherein the first location corresponds to a finger of the examinee.

3. The blood pressure measuring device of claim 2, wherein the first heart rate measuring module includes:
a first camera for imaging the first location to obtain a first image; and
a first heart rate calculation unit for calculating the first heart rate of the examinee based on the first image imaged from the first camera.

4. The blood pressure measuring device of claim 3, wherein the first heart rate calculation unit is configured to extract a green-channel image from the first image imaged from the first camera, and to calculate the first heart rate of the examinee based on the extracted green-channel image.

5. The blood pressure measuring device of claim 2, wherein the first heart rate measuring module is disposed on the finger of the examinee, wherein the first heart rate measuring module is configured to irradiate light of a predetermined frequency to the finger of the examinee, to collect light beams reflected from or transmitted through the finger of the examinee, and to calculating the first heart rate of the examinee based on the collected light beams.

6. The blood pressure measuring device of claim 1, wherein the second heart rate measuring module includes:
a second camera for imaging the second location to obtain a second image; and
a second heart rate calculation unit for calculating the second heart rate of the examinee based on the second image imaged from the second camera.

7. The blood pressure measuring device of claim 6, wherein the second location corresponds to a facial portion of the examinee.

8. The blood pressure measuring device of claim 6, wherein the second heart rate calculation unit is configured to calculate the second heart rate of the examinee based on the second image,
wherein the second heart rate calculation unit includes an image processing module for calculating a skin color change amount from the facial image, and for measuring a blood flow change signal and a blood flow change image from the calculated skin color change amount,
wherein the image processing module includes:
a down-sampling unit for down-sampling the facial image;
a correction unit for carrying out HSV correction of the down-sampled facial image;
an adaptive filtering unit for minimizing motion artifacts in the skin color change amount by updating a weight;
an up-sampling unit for deriving the blood flow change image after the facial image is subjected to the adaptive filtering; and
an averaging unit for deriving the blood flow change signal after the facial image is subjected to the adaptive filtering.

9. The blood pressure measuring device of claim 1, wherein the blood pressure estimation module includes:
a time calculation unit for calculating a pulse transit time between the first and second locations based on the first and second heart rates provided by the first and second heart rate measuring modules;
a velocity calculation unit for calculating a pulse wave velocity based on the pulse transit time calculated via the time calculation unit and, information on the body of the examinee; and
a blood pressure estimation unit for estimating a blood pressure of the examinee based on the pulse wave velocity calculated via the velocity calculation unit.

10. The blood pressure measuring device of claim 1, further comprising a main body, wherein the first heart rate measuring module includes a first camera for imaging the first location, and the second heart rate measuring module includes a second camera for imaging the second location, wherein the first and second cameras are disposed on the main body.

11. The blood pressure measuring device of claim 10, wherein the first location corresponds to a finger of the examinee and the second location corresponds to a facial portion of the examinee,
wherein the second camera is disposed on a front face of the main body, and the first camera is disposed on a rear face of the main body such that the first and second locations are simultaneously imaged by the first and second cameras when the examinee grips the main body by a hand thereof.

12. The blood pressure measuring device of claim 11, further comprising:
a display disposed on the front face of the main body for displaying thereon blood pressure data of the examinee calculated from the blood pressure estimation module; and
a communication module disposed in the main body for transmitting the blood pressure data of the examinee calculated from the blood pressure estimation module to a management server and a terminal of a caregiver of the examinee.

13. A blood pressure measuring method comprising:
measuring a first heart rate at a first location of an examinee's body;
measuring a second heart rate at a second location of the examinee's body, wherein the first location is different from the second location, wherein a first spacing between the first location and a heart of the examinee is different from a second spacing between the second location and the heart of the examinee; and
estimating a blood pressure of the examinee based on the first and second heart rates.

14. The blood pressure measuring method of claim 13, wherein measuring the first heart rate includes:
imaging the first location using a first camera to obtain a first image; and
calculating the first heart rate of the examinee based on the obtained first image.

15. The blood pressure measuring method of claim 14, wherein calculating the first heart rate of the examinee includes:
extracting from the first image a first ROI (region of interest) image for the first heart rate calculation;
extracting a first green-channel image from the first ROI the image to form a first analyzed image; and
calculating the first heart rate of the examinee based on the first analyzed image.

16. The blood pressure measuring method of claim 15, further comprising filtering a noise from the first analyzed image, wherein the filtering occurs between extracting the first green-channel image and calculating the first heart rate.

17. The blood pressure measuring method of claim 13, wherein measuring the second heart rate includes:
imaging the second location using a second camera to obtain a second image; and
calculating the second heart rate of the examinee based on the obtained second image.

18. The blood pressure measuring method of claim 17, wherein calculating the second heart rate of the examinee includes:
extracting from the second image a second ROI (region of interest) image for the second heart rate calculation;
extracting a second green-channel image from the second ROI the image to form a second analyzed image; and
calculating the second heart rate of the examinee based on the second analyzed image.

19. The blood pressure measuring method of claim 18, further comprising filtering a noise from the second analyzed image, wherein the filtering occurs between extracting the second green-channel image and calculating the second heart rate.

20. The blood pressure measuring method of claim 13, wherein estimating the blood pressure includes:
calculating a pulse transit time between the first and second locations based on the first and second heart rates;
calculating a pulse wave velocity based on the pulse transit time, and information on the body of the examinee; and
estimating a blood pressure of the examinee based on the pulse wave velocity.
